(19) United States
(12) Patent Application Publication
(10) Pub. No.: US 2007/0270734 A1
(43) Pub. Date: Nov. 22, 2007

(54) Urinary tract catheter

(76) Inventor: William E. Crisp, Paradise Valley, AZ (US)

Correspondence Address: TOD R NISSLE, PO BOX 55630, PHOENIX, AZ, 85078, US

(21) Appl. No.: 11/653,183

(22) Filed: Jan. 12, 2007

Related U.S. Application Data
(63) Continuation-in-part of application No. 10/406,329, filed on Apr. 3, 2003, now abandoned.

(51) Int. Cl. A61M 1/00

(52) U.S. Cl. 604/21; 604/265; 604/544

(57) ABSTRACT

A urinary catheter is provided that reduces the likelihood of infection. The catheter utilizes a balloon that is positioned in the bladder to prevent removal of the catheter. The balloon, when inflated, takes on a tapered configuration that reduces pressure on the trigone. The catheter incorporates one-way valves which function to make the catheter a close system that minimizes the risk of infection. Spaced apart concentrations of silver and zinc are incorporated on the inner and outer surfaces of the catheter to further reduce the risk of infection.

This is a continuation-in-part of application Ser. No. 10/406,329, filed Apr. 3, 2003.

This invention pertains to a catheter.

More particularly, the invention pertains to an indwelling urinary tract catheter system that minimizes the likelihood that an infection will occur in a patient as the result of insertion or subsequent operation of the catheter in the patient.

In a further respect, the invention pertains to an indwelling urinary tract catheter system that provides increased patient comfort when the catheter is inserted in the urinary tract and that facilitates insertion of the catheter by a nurse.

In a male, the preprostatic urethra and proximal part of the prostatic urethra are lined with the urothelial type epithelium. This urothelial epithelium is continuous with linings of the seminal vesicles and with prostate ducts and bulbourethral gland ducts, The continuity of the urothelial epithelium with the seminal vesicle linings and ducts of the prostate and bulbourethral gland contributes to the spread of urinary tract infections when the mucous membrane barrier is compromised by a conventional catheter.

Below the openings of the ejaculatory ducts, the epithelium lining is a patchily pseudostratified or stratified columnar type. Such stratified epithelium lines the membranous urethra and most of the penile urethra. Mucous-secreting cells frequently occur in the stratified epithelium and there are numerous recesses connected to mucous glands.

The mucous membrane lining the female urethra comprises stratified epithelium and a supporting layer of connective tissue. The connective tissue is called the lamina propria and includes elastic fibers. The fibers are in an orientation extending both longitudinally and circularly around the urethra.

The internal urethral orifice opens into the inner bladder adjacent the trigone. The trigone includes smooth muscle. The smooth muscle has two layers. The superficial muscle comprises the first layer. The deep detrussor muscle comprises the second layer. The deep trigonal muscle is the postero-inferior portion of the detrusor muscle. The superficial muscle is comprised of small diameter muscle bundles. These muscle bundles are continuous proximally with muscle bundles of the intramural ureters. The superficial muscle is thin but thickens at its superior border to form the interureteric crest. Comparable thickenings form along the lateral edges of the superficial muscle. The ureters interconnect the bladder and kidneys.

When a conventional catheter, for example a Foley catheter, is inserted into the bladder of an individual of either gender, the catheter extends upwardly through the urethra and the internal urethral orifice into the bladder. The distal end of the catheter includes an opening and an inflatable balloon portion located beneath the opening. The opening functions to permit urine to drain from the bladder through a channel in the catheter and into a collection bag at the proximate end of the catheter. The balloon is inflated after the distal end is initially inserted through the urethra and the internal urethral orifice into the bladder. The inflated balloon seats on the trigone. Since the Foley catheter and other comparable catheters typically remain in and inhabit the bladder and urethra for extended periods of time, they are termed indwelling urinary catheters.

Indwelling urinary catheters account for over 30% of the morbidity in hospitals and nursing homes. Consequently, existing indwelling catheters dramatically increase the cost of medical care, and also increase for a patient the risk of illness and death.

One cause of infection by an indwelling catheter is the introduction into a patient of a microbe or other disease causing component during the insertion of the catheter into the urethra and bladder. The microbe typically exists on the external surface of the catheter due to improper sterilization, due to handling by the care giver, or due to other causes.

Another cause of infection by an indwelling catheter is the introduction into a patient of a microbe that ascends superiorly upwardly along the exterior surfaces of the catheter through the urethra and into the bladder.

The other etiology of infection by an indwelling catheter is the introduction into a patient of a microbe through the channel that extends through the interior of the catheter and through which urine drains from the distal end of the catheter to the collection bag at the proximate end of the catheter.

Infections of the bladder can cause serious complications, especially for elderly patients, for patients having a weakened immune system, or for patients having preexisting diseases or weakened renal—bladder systems.

As noted, in the male urethra, the proximity of urothelial epithelium to the seminal vesicle epithelium and ducts of the prostate and bulbourethral gland contributes to the spread of urinary tract infections, and can result in infection of the prostate and testes.

Infections can also spread via the urethra to the bladder and on to the kidneys. If an infection originating in the bladder spreads to the kidneys, pyelonephritis can result. Pyelonephritis is inflammation of the kidney affecting the glomerulus or parenchyma.

Any infection in the lower urinary system has the potential of infecting the kidneys causing pyelonephritis which is a serious complication with potential dire consequences. Infection in the kidney (pyelonephritis) can cause nephrosclerosis with atrophy and renal failure (uremia) and/or hypertension.

Accordingly, it is highly desirable to provide a urinary tract catheter that reduces the possibility of infection.

Therefore, it is a principal object of the invention to provide an improved method and apparatus for removing urine from the bladder of a patient into a storage container with minimal risk for infection.

A further object of the invention is to provide an improved urine removal method and apparatus that reduces the likelihood that microbes or other disease causing components will be introduced into the body of a patient during catheter insertion and use.

These and other, further and more specific objects and advantages of the invention will be apparent from the following detailed description of the invention, taken in conjunction with the drawings, in which:

FIG. 1 is a front perspective partial section view illustrating a bladder;

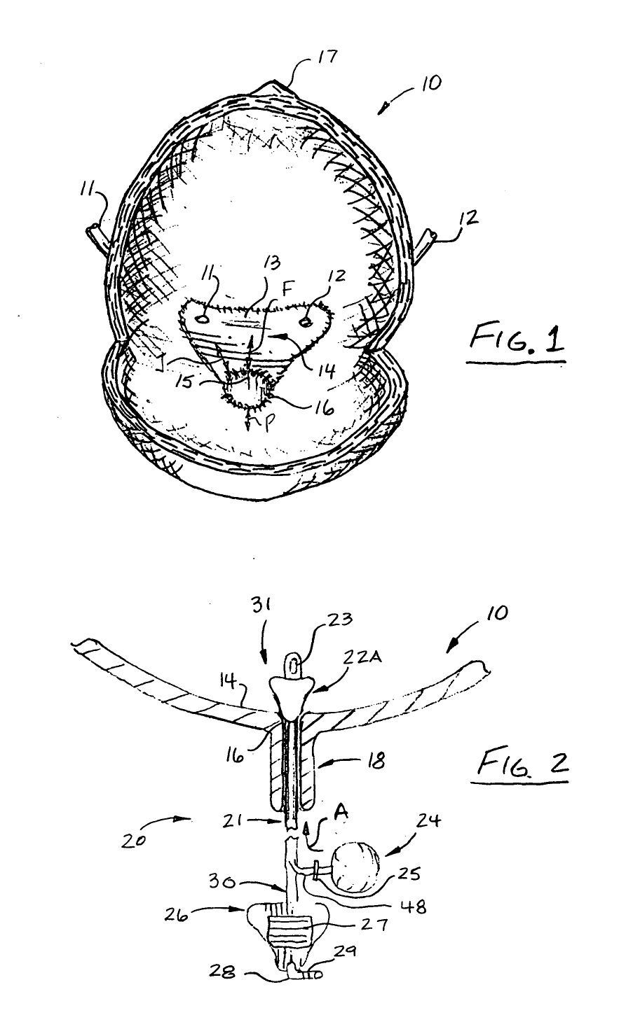

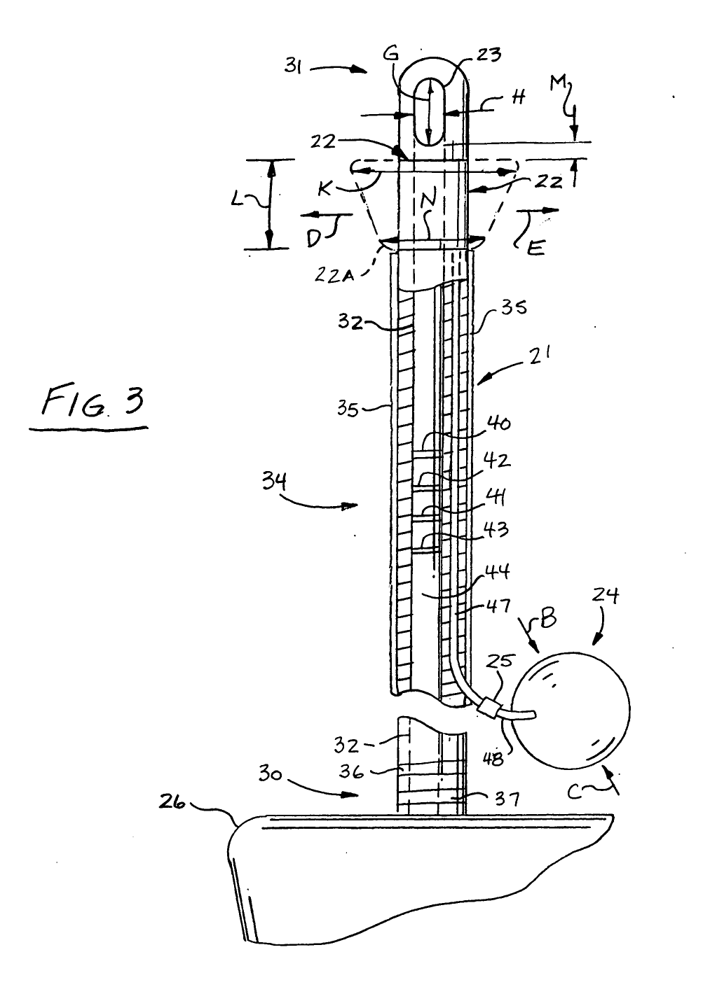

FIG. 2 is a front elevation view illustrating a catheter constructed in accordance with the principles of the invention inserted in the urethra and bladder of a patient; and, FIG. 3 is a front elevation view of the catheter of FIG. 2 illustrating additional construction details thereof.

Briefly, in accordance with my invention, I provide an improved urinary catheter system for a bladder. The bladder includes a trigone. The catheter system comprises a urine collection container; an inflation system; and, an elongate pliable tube. The tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and a balloon positioned beneath said opening and inflatable into a tapered configuration to minimize the contact surface area of the trigone; includes a first channel connecting the opening and the collection container; includes a second channel interconnecting the balloon and the inflation system such that the inflation system can be used to inflate the balloon into the tapered configuration; and, also includes an intermediate section positioned in the urethra when the distal end is inserted in the bladder.

In another embodiment of the invention, I provide an improved urinary catheter system for a bladder. The bladder includes a trigone. The improved catheter system comprising a urine collection container; an inflation system; and, an elongate pliable tube. The pliable tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening; has a first channel connecting the opening and the collection container; has a second channel interconnecting the balloon and the inflation system such that the inflation system can be used to inflate the balloon, and, has an intermediate section positioned in the urethra when the distal end is inserted in the bladder, the intermediate section including an outer foam layer.

In a further embodiment of the invention, I provide an improved urinary catheter system for a bladder. The bladder includes a trigone. The catheter system comprises a urine collection container; an inflation system; and, an elongate pliable tube. The pliable tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and an inflatable balloon positioned beneath the opening; includes a first channel connecting the opening and the collection container; includes a second channel interconnecting the balloon and the inflation system such that the inflation system can be used to inflate the balloon; includes an intermediate section positioned in the urethra when the distal end is inserted in the bladder; includes at least one source of silver in the first channel; and, includes at least one source of zinc in the first channel and spaced apart from the source of silver.

In still another embodiment of the invention, I provide an improved urinary catheter system for a bladder. The bladder includes a trigone. The catheter system comprises a urine collection container; an inflation system; and, an elongate pliable tube. The tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening; has a first channel connecting the opening and the collection container; has a second channel interconnecting the balloon and the inflation system such that the inflation system can be used to inflate the balloon; has an intermediate section positioned in the urethra when the distal end is inserted in the bladder; has an exterior surface; has at least one source of silver on the exterior surface; and, has at least one source of zinc on the exterior surface spaced apart from the source of silver.

In still a further embodiment of the invention, I provide an improved urinary catheter system for a bladder. The bladder includes a trigone. The catheter system includes a urine collection container; an inflation system including a sealed reservoir of liquid; and, an elongate pliable tube. The elongate pliable tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening; has a first channel connecting the opening and the collection container; has a second channel interconnecting the balloon and the inflation system such that the inflation system can be used to inflate the balloon; and includes an intermediate section positioned in the urethra when the distal end is inserted in the bladder. The catheter system also includes apparatus for pressurizing the liquid to displace the liquid from the sealed reservoir through the second channel into the balloon to inflate the balloon.

In yet a further embodiment of the invention, I provide an improved urinary catheter system for a bladder. The bladder includes a trigone. The catheter system comprises a urine collection container; an inflation system; and, an elongate pliable tube. The pliable tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and an inflatable balloon positioned beneath the opening; includes a first channel connecting the opening and the collection container; includes a second channel interconnecting the balloon and the inflation system such that the inflation system can be used to inflate the balloon; includes an intermediate section positioned in the urethra when the distal end is inserted in the bladder; and, includes an exterior self-lubricating surface.

In yet another embodiment of the invention, I provide an improved urinary catheter system for a bladder. The bladder includes a trigone. The catheter system comprises a urine collection container; an inflation system; and, an elongate pliable tube. The pliable tube has a proximate end extending to the urine collection container and located exterior of the urethra when the distal end is inserted in the bladder; has a distal end shaped and dimensioned to be inserted through a urethra into the bladder and including an opening for draining urine from the bladder, and an inflatable balloon positioned beneath the opening, the opening extending less than two centimeters above said balloon; includes a first channel connecting the opening and the collection container; includes a second channel interconnecting the balloon and the inflation system such that the inflation system can be used to inflate the balloon; and, includes an intermediate section positioned in the urethra when the distal end is inserted in the bladder.

In still yet a further embodiment of the invention, I provide an improved urinary catheter system for a bladder. The bladder includes a trigone. The catheter system comprises a urine collection container including a one-way valve for emptying the container; includes an inflation system; and, includes an elongate pliable tube. The tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and an inflatable balloon positioned beneath the opening; includes a first channel connecting the opening and the collection container; includes a second channel interconnecting the balloon and the inflation system such that the inflation system can be used to inflate the balloon; and, includes an intermediate section positioned in the urethra when the distal end is inserted in the bladder.

In still yet another embodiment of the invention, I provide an improved urinary catheter system for a bladder. The bladder includes a trigone. The catheter system comprises a tapered urine collection container; an inflation system; and, an elongate pliable tube. The pliable tube includes a proximate end extending to the urine collection container and located exterior of the urethra when the distal end is inserted in the bladder; includes a distal end shaped and dimensioned to be inserted through a urethra into the bladder and including an opening for draining urine from the bladder, and a balloon positioned beneath the opening; includes a first channel connecting the opening and the collection container; includes a second channel interconnecting the balloon and the inflation system such that the inflation system can be used to inflate the balloon; and, includes an intermediate section positioned in the urethra when the distal end is inserted in the bladder.

In a further embodiment of the invention, I provide an improved urinary catheter system for a bladder. The bladder includes a trigone. The catheter system comprises a urine collection container including a light reflective scale; an inflation system; and, an elongate pliable tube. The pliable tube has a proximate end extending to the urine collection container and located exterior of the urethra when the distal end is inserted in the bladder; has a distal end shaped and dimensioned to be inserted through a urethra into a bladder and including an opening for draining urine from the bladder, a balloon positioned beneath the opening; has a first channel connecting the opening and the collection container; has a second channel interconnecting the balloon and the inflation system such that the inflation system can be used to inflate the balloon; and, has an intermediate section positioned in the urethra when the distal end is inserted in the bladder.

In another embodiment of the invention, I provide an improved urinary catheter system for a bladder. The bladder includes a trigone. The improved catheter system comprises a urine collection container; an inflation system; and, an elongate pliable tube. The tube has a proximate end extending to the urine collection container and located exterior of the urethra when the distal end is inserted in the bladder; a distal end shaped and dimensioned to be inserted through a urethra into a bladder and including an opening for draining urine from the bladder; a first channel connecting the opening and the collection bag; an intermediate section positioned in the urethra when the distal end is inserted in the bladder, the intermediate section including an exterior surface comprised of hydrophilic foam.

Turning now to the drawings, which depict the presently preferred embodiments of the invention for the purpose of illustrating the practice thereof and not byway of limitation of the scope of the invention, and in which like reference characters refer to corresponding elements throughout the several views, FIG. 1 illustrates a bladder, generally indicated by reference character 10. Bladder 10 includes ureters 11 and 12. Each ureter interconnects bladder 10 with a kidney (not shown). Trigone 14 is where the ureters 11 and 12 empty into the bladder and is adjacent the top of the urethra and the urethral orifice 16. The top of the urethra (i.e., the urethral wall) circumscribes the urethral orifice 16 and is intermediate the trigone and urethral orifice 16. Trigone 14 includes interureteric crest 13. Bladder 10 also includes uvula 15 and urethral orifice 16. The male urethra extends from the internal urethral orifice in the urinary bladder to the external urethral orifice at the end of the penis.

FIGS. 2 and 3 illustrate an indwelling urinary catheter system, generally indicated by reference character 20, inserted in the urethra 18 and bladder 10 of a patient. System 20 includes elongate pliable tube 21 fabricated from rubber or from some other polymer or other desired material. The distal end 31 of tube 21 include tapered, generally conically shaped inflated balloon 22A and opening 23. Urine drains from bladder 10 through opening 23 and through channel 32 into a urine collection bag 26 or other collection container. Proximate end 30 extends to bag 26. Bag 26 is tapered and, like inflated balloon 22A, generally has the shape of an inverted cone.

The tapered configuration of inflated balloon 22A is important in the practice of the invention because the lower portion of inflated balloon 22A contact and presses against a smaller area of the trigone than do the spherical balloons used in conventional urinary catheters. Further inflated balloon 22A has a height of less than two centimeters, preferably in the range of 0.5 centimeter to about 1.9 centimeters. This height is less than the inflated height of balloons found in conventional catheters, which conventional balloons typically have a height of two to three centimeters. The width of the top of inflated balloon 22A is indicated by arrows K in FIG. 3 and is preferably less than two centimeters. The width of the bottom of inflated balloon 22A is indicated by arrows N in FIG. 3 and is about equal to the diameter of drainage tube 21. While the diameter of tube 21 can vary as desired, such diameter presently is about six millimeters.

The height of uninflated balloon 22 is about equal to the height of inflated balloon 22A. This height presently is less than two centimeters. The top of balloon 22 and of inflated balloon 22A is, as indicated by arrows M in FIG. 3, about one-eighth of an inch below the bottom of opening 23. The distance of balloon 22 below the bottom of opening 23 can vary as desired, but preferably is in the range of one-eighth of an inch to one-quarter of inch.

The tapered configuration of collection bag 26 is important in the practice of the invention because the shape of bag 26 helps to insure that the bag drains completely when one-way valve 29 and tube 28 are utilized to drain bag 26 into another container. The collection bag 26 can have spaced apart concentrations of silver and zinc on the inside of the bag to prevent ascending infection. Urine drains from bag 26 through tube 28 and one-way valve 29 under gravity. A two-way valve or any other desired valve system can be incorporated in bag 26. A one-way valve is preferred, however, because it reduces the likelihood that microbes will enter bag 26 and travel up channel 32 and escape into the bladder 10 and urethra 18. Similarly, an inflation system other than the liquid filled resilient orb 24 can be utilized in the practice of the invention. Orb 24 is, however, preferred because it is sealed and reduces the likelihood microbes will travel up the catheter into the bladder 10.

The exterior surface 33 of tube 21 is preferably, but not necessarily, comprised of a self-lubricating or smooth material so that K-Y jelly or other lubricants are not necessary when tube 21 is inserted in the urethra and bladder of a patient, thereby avoiding another potential source of infection. By way of example, and not limitation, surface 33 can be comprised of teflon or polypropylene. A portion 34 of tube 21 intermediate the distal 31 and proximate 30 ends of tube 21 is preferably provided with an exterior hydrophilic foam coating 35 that softly engages the internal mucous membrane of urethra 18. Further, coating 35 preferably is impregnated, coated, or otherwise supplied with spaced apart concentrations of silver and zinc. The inside of tube 21 can also be impregnated, coated, or otherwise supplied with spaced apart concentrations of silver and zinc. The distance between these concentrations can vary as desired, but is typically in the range of 0.25 mm to 4.0 mm, preferably 0.5 to 1.5 mm. When coating 35 contacts bodily fluids, an electrical current arises between the silver and zinc concentrations. This current attracts microbes to the silver concentrations. When the microbes come into contact with the silver concentrations the microbes are killed.

All bacteria and other pathogenic organisms carry a negative charge. When the bimetallic pattern of dissimilar metals is moistened by body fluids (urine, mucous, etc.) A current flow is induced that draws the bacteria and other pathogenic organisms to the positively charged silver anode where the organisms are destroyed. The silver attaches to the sulfhydril groups and denatures the protein complexes vital to respiration and other functions.

In FIG. 3, a strip 36 of silver is shown spaced apart from a strip 37 of zinc on the exterior surface of tube 21. Similar strips can be formed on or in foam coating 35. Similar spaced apart strips of silver 40, 41 and zinc 42, 43 can be formed on the interior surface 44 of channel 32. The concentrations of silver and zinc can take on desired patterns or forms other than strips. For example, patterns of spaced apart dots, squares, etc. can be utilized. Metals other than silver and zinc can, if desired, be utilized to produce electrical current when tube 21 contacts bodily fluids. Silver and zinc are presently preferred because they produce a microbiocidal wet battery that does not promote the evolution of resistant organisms. Pathogenic organisms do not develop resistance to silver as they do to antibiotics.

The smooth outer surface of a conventional catheter flattens the inner surface of the urethra and makes it difficult for glands along the urethra to excrete moisture to the urethral lining. Further, natural movement of the urethra permits air to move into the urethra between the catheter tube and the urethral lining and to dry the mucosa. The foam coating 35 used in the catheter of the invention helps to avoid such problems and to avoid irritation of the urethral meatus. First, the foam coating absorbs moisture and swells. The moisture in the foam functions to moisturize the urethral lining to reduce the likelihood the mucosa will dry. Second, when the hydrophillic foam coating swells it prevents air from entering the urethra and drying the mucous membrane. The foam is also coated or impregnated with interspersed concentrations of silver and zinc for the prevention of infection. Third, since the surface of the foam is not continuous but comprises a plurality of openings circumscribed by wall structures (much like the hexagonal openings in a honeycomb are circumscribed by wall structures), the foam produces a plurality of point or edge contacts and permits portions of the mucosa to extend partially or completely into openings in the foam. This in essence increases the mucosa surface area contacted by the foam and facilitates the transfer of moisture from the foam to the mucosa or vice-versa. Fourth, the point-edge contact of the foam with the mucosa functions to form seals that further hinder the travel of air into the urethra intermediate tube 21 and the inner lining of the urethra. Coating 35 can consist of any hydrophilic foam or other material that produces the above-described edge or point contacts and includes pores that increase the surface area contact between the coating 35 and mucosa in the urethra. The foam is preferably, but not necessarily, resilient.

In use, a catheter system 20 is provided. The balloon 22 is not inflated. The exterior of tube 21 is self-lubricated, either because of the self-lubricating characteristics of the material used to fabricate tube 21, or because of a lubricating or friction reducing coating or material (i.e., Teflon) applied to the exterior of tube 21. Tube 21 is inserted through urethra 18 until distal end 31 is at the position illustrated in FIG. 2. Orb 24 is manually squeezed and compressed in the directions indicated by arrows B and C in FIG. 3, or apparatus other than a user's fingers is used, to force fluid through one-way valve 25 and through channel 47 in the direction of arrow A to fill (i.e., inflate) balloon 22 and cause it to expand in the directions of arrows D and E from the stored, uninflated configuration 22 depicted in FIG. 3 to the expanded, tapered configuration indicated by dashed lines 22A in FIG. 3 and illustrated in FIG. 2. When hollow orb 24 is compressed, a saline solution or other liquid in orb 24 is forced through one-way valve 25 and conduit 48 to channel 47. Valve 25 prevents liquid in balloon 22 or channel 47 from flowing back into orb 24. The use of orb 24 to hydraulically fill balloon 22 avoids requiring a nurse to fill balloon 22 in conventional fashion with a syringe. The use of such a syringe is a frequent cause of infection.

After balloon 22 is inflated to its expanded, tapered configuration 22A, installation of the catheter system 20 is complete. Urine that accumulates in bladder 10 flows through opening 23 and channel 32 into bag 26. Bag 26 is periodically emptied using conduit 28 and one-way valve 29. The catheter is removed by first cutting the catheter to release the pressurized fluid from the inflated balloon 22A, causing the balloon to deflate. The catheter is then carefully removed from the urethra.

The shape and dimension of opening 23 can vary as desired. It is presently preferred however, that opening 23 be about five millimeters high, as indicated by arrows G, and three millimeters wide, as indicated by arrows H. A conventional urinary catheter includes an opening that is five millimeters high and two and one-half millimeters wide. The addition width H in the catheter system of the invention is important in reducing the likelihood that opening 23 will be blocked.

Graduated markings 27 can be provided on bag 26 or on any other urine collecting container utilized in the invention. The markings 27 indicated the volume of urine in bag 26 or indicate what proportion of bag 26 is filled or remains to be filled. Markings 27 can be light reflective to facilitate readings the markings. Markings 27 can also fluoresce. The catheter of the invention can be fabricated without balloon 22, orb 24, and channel 47. Either this “balloon-less” catheter or a catheter of the type illustrated in the drawings can be used to drain an abscess or remove fluid from other body cavities.

The shape and dimension of balloon 22 and inflated balloon22A can vary as desired. However, one significant advantage of the balloon 22 of the invention is that when it is inflated, the lower portion of balloon 22 preferably rests and seats on top of the urethra without contacting the trigone, or, only contacting a relatively small portion of the trigone. The lower portion of the inflated balloon 22A can contact both the top of the urethra and the trigone. The lower portion of the inflated balloon 22A can also contact or extend adjacent a portion of the upper inner surface of the urethral wall that is adjacent the top of the urethra. The area of the trigone contacted by the bottom of the balloon is preferably less than the area of the top of the urethra contacted by the bottom of the balloon, or, is preferably less than the sum total of the surface area of the top of the urethra and of the upper inner surface of the urethral wall contacted by the bottom of the balloon. In another embodiment of the invention, the area of the trigone contacted by the bottom of the balloon is (1) 50% of the sum total of the surface area of the top of the urethra contacted by the bottom of the balloon 22A and the surface area of the trigone contacted by the bottom of the balloon, or, (2) 50% of the sum total of the surface area of the top of the urethra contracted by the bottom of the balloon 22A, of the surface area of the upper inner surface of the urethral wall contacted by the bottom of the balloon 22A, and, of the surface area of the trigone contacted by the bottom of the balloon. Most preferably, the bottom of the inflated balloon 22A normally does not contact the trigone at all.

In another embodiment of the invention, the surface area of the top of the urethra and/or of the trigone that is contacted by the bottom of the inflated balloon and is adjacent and extends outwardly from the perimeter of internal orifice 16 in any direction, as indicated by arrows J and K and P, extends outwardly no more than one millimeter away from the perimeter of the internal orifice 16. Consequently, since the top of the urethral wall that circumscribes orifice 16 may be at least one millimeter wide, if the surface area of tissue around orifice 16 that is contacted by the bottom of the inflated balloon extends outwardly from orifice 16 a distance of one millimeter or less, then the balloon may not contact any portion of the trigone.

In a further embodiment of the invention, the surface area of the top of the urethra and/or of the trigone that is contacted by the bottom of the inflated balloon and is adjacent and extends outwardly from the perimeter of internal orifice 16 in any direction, as suggested by arrows J and K and P, extends outwardly no more than two millimeters away from the perimeter of the internal orifice 16. Consequently, since the top of the urethral wall that circumscribes orifice 16 may be at least two millimeters wide, if the surface area of tissue around orifice 16 that is contacted by the bottom of the inflated balloon extends outwardly from orifice 16 a distance of two millimeters or less, then the balloon may not contact any portion of the trigone.

In still a further embodiment of the invention, the surface area of the top of the urethra and/or of the trigone that is contacted by the bottom of the inflated balloon and is adjacent and extends outwardly from the perimeter of internal orifice 16 in any direction, as suggested by arrows J and K and P, extends outwardly no more than three millimeters away from the perimeter of the internal orifice 16.

In still another further embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than four millimeters away from the perimeter of the internal orifice 16.

In yet a further embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than five millimeters away from the perimeter of the internal orifice 16.

In yet another embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than six millimeters away from the perimeter of the internal orifice 16.

In yet still a further embodiment of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than seven millimeters away from the perimeter of the internal orifice 16.

In yet still other embodiments of the invention, the surface area of the top of the urethra and/or of the trigone that is contacted by the bottom of the inflated balloon and is adjacent and extends outwardly from internal orifice 16 in any direction, as suggested by arrows J and K and P, extends outwardly no more than eight, nine, ten, eleven, twelve, thirteen, fourteen, fifteen, sixteen, seventeen, eighteen, nineteen or twenty millimeters away from the perimeter of the internal orifice 16, as the case may be and as may be desirable depending on the physiology of the individual.

The lower portion of inflated balloon 22A can be seated on top of the urethra because the urethra is normally contracted at least in part and because the top of the urethra therefore forms a support surface or area on which the lower portion of balloon 22A can rest. Such a support surface exists even when tube 21 is in the urethra. The support surface circumscribes tube 21.

Having described my invention in such terms as to enable those of skill in the art to make and practice it, and having described the presently preferred embodiments thereof, I Claim:

1. A urinary catheter system for a bladder including a trigone, an internal orifice, and a urethra, the urethra having a top located in the bladder, said catheter system comprising
(a) a urine collection container;
(b) an inflation system;
(c) an elongate pliable tube having
(i) a distal end shaped and dimensioned to be inserted through a urethra into a bladder and including
an opening for draining urine from the bladder,
a balloon positioned beneath said opening and inflatable into a tapered configuration shaped and dimensioned such that the surface area of the trigone contacted by said balloon is less than the surface area of the top of the urethra contacted by said balloon,
(ii) a proximate end extending to said urine collection container and adapted to be located exterior of the urethra when said distal end is inserted in the bladder,
(iii) a first channel connecting said opening and said collection container,
(iv) a second channel interconnecting said balloon and said inflation system such that said inflation system can be used to inflate said balloon into said tapered configuration, and,
(v) an intermediate section adapted to be positioned in the urethra when said distal end is inserted in the bladder.

2. The system of claim 1 including
(a) at least one source of silver in said first channel; and,
(b) at least one source of zinc in said first channel and spaced apart a selected distance from said source of silver, said selected distance adapted to cause, without use of an external power source, an electrical current to arise between said source of zinc and said source of silver when bodily fluid contacts said sources of zinc and silver, said electrical current attracting microbes to said source of silver to kill said microbes.

3. The system of claim 1 wherein said balloon when inflated does not contact the trigone.

4. The system of claim 1 wherein said balloon when inflated has a top and tapered bottom.

5. The system of claim 1 wherein said balloon when inflated contacts an area of the trigone substantially equivalent to the area of the top of the urethra contacted by the balloon.

6. The system of claim 1 wherein the surface area of the trigone contacted by said inflated balloon is greater than the area of the top of the urethra contacted by said balloon and extends outwardly from the perimeter of the inner orifice no more than one millimeter.

7. The system of claim 6 wherein the surface area of the trigone contacted by said inflated balloon extends outwardly from the perimeter of the inner orifice no more than two millimeters.

8. The system of claim 6 wherein the surface area of the trigone contacted by said inflated balloon extends outwardly from the perimeter of the inner orifice no more than three millimeters.

9. The system of claim 6 wherein the surface area of the trigone contacted by said inflated balloon extends outwardly from the perimeter of the inner orifice no more than four millimeters.

10. The system of claim 6 wherein the surface area of the trigone contacted by said inflated balloon extends outwardly from the perimeter of the inner orifice no more than five millimeters.

11. The system of claim 6 wherein the surface area of the trigone contacted by said inflated balloon extends outwardly from the perimeter of the inner orifice no more than six millimeters.

12. The system of claim 6 wherein the surface area of the trigone contacted by said inflated balloon extends outwardly from the perimeter of the inner orifice no more than seven millimeters.

13. The system of claim 6 wherein the surface area of the trigone contacted by said inflated balloon extends outwardly from the perimeter of the inner orifice no more than eight millimeters.

14. The system of claim 6 wherein the surface area of the trigone contacted by said inflated balloon extends outwardly from the perimeter of the inner orifice no more than nine millimeters.

15. The system of claim 6 wherein the surface area of the trigone contacted by said inflated balloon extends outwardly from the perimeter of the inner orifice no more than ten millimeters.

16. The system of claim 6 wherein the surface area of the trigone contacted by said inflated balloon extends outwardly from the perimeter of the inner orifice no more than eleven millimeters.

17. The system of claim 6 wherein the surface area of the trigone contacted by said inflated balloon extends outwardly from the perimeter of the inner orifice no more than twelve millimeters.

18. The system of claim 6 wherein the surface area of the trigone contacted by said inflated balloon extends outwardly from the perimeter of the inner orifice no more than thirteen millimeters.
